(19) United States
(12) Patent Application Publication
(10) Pub. No.: US 2009/0202642 A1
(43) Pub. Date: Aug. 13, 2009

(54) Drug Delivery System Comprising Microparticles and Gelation System

(76) Inventors: Xiao Huang, Austin, TX (US); Jizong Gao, Cedar Park, TX (US); Jian Yao, Austin, TX (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/367,948

(22) Filed: Feb. 9, 2009

Related U.S. Application Data
(60) Provisional application No. 61/027,276, filed on Feb. 8, 2008.

(51) Int. Cl. A61K 9/12; A61K 9/00; A61K 9/14; A61K 39/395; A61K 31/7008; A61K 31/715; A61K 31/728

(52) U.S. Cl. 424/488; 424/400; 424/499; 424/158.1; 514/62; 514/54

(57) ABSTRACT

Disclosed are drug delivery compositions comprising a continuous aqueous phase comprising a reverse thermal gelation system comprising a blend of a cellulose derivative and polyethylene glycol; a discontinuous particulate phase comprising microparticles; and an agent to be delivered contained in at least said discontinuous particulate phase. Also disclosed are sustained release compositions formed using the drug delivery compositions and methods of using those compositions.

### BACKGROUND OF THE INVENTION

1. Field Of The Invention

The invention relates to bioactive agent delivery systems. In particular, it relates to such systems that permit sustained and controlled release of the agent within a defined environment, typically an in vivo environment.

2. Description of the Related Art

Many biologically active macro-molecules such as peptides/proteins and DNA, effective for gene therapy and a variety of therapeutic applications, have become commercially available through advances in recombinant DNA and other technologies. However, these molecules are limited to parenteral administration due to their susceptibility to degradation in the gastrointestinal tract. Treatment for chronic illnesses or indications may require multiple injections per day over many days, or months. Patient compliance is usually poor. Treatment of injuries to joints and other organs frequently requires localized, long term delivery of an agent. Administration of such agents to localized areas in a manner that provides sustained and controlled release in a patient is typically difficult.

Attempts to maintain a steady level of medication using biodegradable polymers have recently attracted considerable attention. Because, these polymers are biodegradable, they do not require retrieval after the medication is exhausted. Therefore, they can be fabricated into microspheres, microcapsules or nanospheres, with the drug encapsulated in them. Various micro-encapsulation techniques for incorporating a bio-active agent into a microparticle carrier are taught in the art.

However, rapid initial release of the drug, commonly referred to as burst release, is often observed immediately after administration of microparticle delivery systems. Release of the agent from a microparticle delivery system comprises an initial burst release from the surface of the device. Much higher than normal therapeutic levels of medication in the blood resulting from the burst effect of a microparticle system are undesirable because they often cause side effects such as nausea, vomiting, delirium and, sometimes, death. Similar situations can occur when the polymer matrix is catastrophically eroded.

### SUMMARY OF THE INVENTION

The invention provides a biodegradable gel matrix and a microparticle system wherein the microparticle is contained within or embedded in the biodegradable gel matrix, from which the bioactive agent is released in a controlled manner. The bioactive agents may be located within the microparticle only, or within both the microparticle and the gel matrix. The gel matrix is formed from a blend of materials that provides surprising strength and durability.

The invention also provides a gel solution or blend that upon administration to a mammal gels or sets to form the gel matrix. In this aspect, the invention provides a dosage form that comprises a drug-containing microparticle delivery system suspended in a reverse thermal gelation (“RTG”) system. Upon administration to the body of a mammal such as a human, the RTG system sets, forming a depot and entrapping the drug-containing microparticles.

Thus, in one aspect, the invention provides a dual phase polymeric agent-delivery composition comprising:
(a) a continuous aqueous phase comprising a reverse thermal gelation system comprising a blend of a cellulose derivative and polyethylene glycol;
(b) a discontinuous particulate phase comprising microparticles; and
(c) an agent to be delivered contained in at least said discontinuous particulate phase.

In another aspect, the invention provides methods for delivering an agent to a mammalian subject in a controlled manner for a sustained period of time. This method involves providing a dual phase polymeric delivery composition as described herein; maintaining the composition as a liquid; administering said composition as a liquid to a confined location in the patient; and permitting the composition to form a gel within the confined location in the patient.

In still another aspect, the invention provides a dual-phase sustained release gelled dosage form. This dosage form comprises a continuous hydrogel phase comprising a blend of a cellulose derivative and polyethylene glycol; a discontinuous particulate phase comprising microparticles; and an agent to be delivered contained in at least said discontinuous particulate phase.

The dual phase delivery composition delivers the agent in a sustained release fashion. The composition further allows for controlled release of the agent over an extended period of time.

The composition of the invention reduces or eliminates the “burst” effect associated with microparticle delivery systems. This, in turn, enhances the length of time over which the agent is delivered, which also leads to improved bioavailability and duration of action.

The compositions of the invention form gels rapidly when administered to a mammal and can suspend the microparticles effectively without plugging of needles used to deliver the composition during administration. The RTG system can be blended to modulate the temperature at which the RTG system will gel, allowing the compositions to be adapted for use in different mammalian systems.

The invention provides drug delivery compositions that can be used to deliver or administer a variety of agents such as pharmaceuticals or bioactive materials. Among the agents that can be delivered using the drug delivery compositions of the invention are small molecule pharmaceuticals such as non-steroidal anti-inflammatory compounds, anti-cancer agents, peptides, proteins, genes, and oligonucleotides.

In a particular aspect, the invention provides methods for treating or repairing a joint in a mammal comprising injecting into a joint space in need of such treatment a dual phase polymeric delivery composition of the invention. By way of example, the invention is particularly well adapted for treating arthritis, such as, for example, osteoarthritis and rheumatoid arthritis.

### BRIEF DESCRIPTION OF THE FIGURE

FIG. 1 is a graph showing chondroitin sulfate (CS) release kinetics from 50:50 PLGA microspheres alone (filled squares) and from 50:50 PLGA microspheres embedded within a MC-PEG hydrogel (filled triangles).

### DETAILED DESCRIPTION OF THE INVENTION

### Definitions

“Biocompatible” shall mean any substance that is suitable for use in an warm-blooded animal or a human body.

“Biodegradable” refers to a material, e.g., a hydrogel or microparticle, that can break down or degrade within the mammalian, preferably human, body to non-toxic products. In the context of the invention, this degradation may take place after or while a bioactive agent has been or is being released.

“Parenteral” shall mean intramuscular, intraperitoneal, intra-abdominal, subcutaneous, and, to the extent feasible, intravenous and intraarterial.

“Bioactive agent”, “bio-active agent” or “agent” shall mean any drug, organic compound, substance, nutrient or biologically beneficial agent including proteins, peptides (including polypeptides and oligopeptides) hormones, vaccines, oligonucleotides, nucleic acids, steroids, antibiotics, antibodies, live cells, tissue derived compositions and other pharmaceutically active agents. Suitable drugs are described in such well-known literature references as the Merck Index, the Physicians Desk Reference, and The Pharmacological Basis of Therapeutics. A brief listing of specific agents is provided for illustration purposes only, and shall not be deemed as limiting: anti-cancer agents such as mitomycin, bleomycin, BCNU, carboplatin, doxorubicin, daunorubicin, methotrexate, paclitaxel, taxotere, actinomycin D and camptothecin; antipsychotics such as olanzapine and ziprasidone; antibacterials such as cefoxitin; anthelmintics such as ivermectin; antivirals such as acyclovir; immunosuppressants such as cyclosporin A (cyclic polypeptide-type agent), steroids, and prostaglandins; and glycosaminoglycans such as chondroitin sulfate, heparan sulfate, dermatan sulfate, hyaluron, heparin, and keratan sulfate.

“Peptide,” “polypeptide,” “oligopeptide” and “protein” shall be used interchangeably when referring to peptide or protein drugs and shall not be limited as to any particular molecular weight, peptide sequence or length, field of bioactivity or therapeutic use unless specifically stated.

“Biological environment” shall mean any environment, whether in vitro or in vivo, where biological activity may be controlled by bioactive agent release. Preferably, the biological environment refers to mammals including humans.

“Microparticles” shall include any particle capable of containing a bio-active agent that is to be released within the body including specialized forms such as microcapsules, microspheres, and nanospheres, and the like, whether natural or artificial.

“Microcapsules,” “Microspheres” and “Nanospheres” refer generally to any highly-engineered and processed microparticle used to contain and release a bio-active agent.

The terms “gel” and “hydrogel” and “hydrogel matrix” mean the semi-solid phase that spontaneously occurs as the conditions are met for gelation of the gel solution.

“Polymeric gel” shall mean any polymer, copolymer, block copolymer and the like that exhibits gelation properties for a period of time when administered within a biological environment, but may be a liquid under conditions not present in that environment.

“Thermosensitive polymeric gel” shall mean any polymeric gel that, depending on temperature, may exist in liquid state or a gel state.

The terms “gelation temperature” or “gel/sol temperature” mean the temperature at which a solution transitions to become a gel.

The term “reverse thermal gelation system” (RTG system) refers to a material or a mixture of materials that exhibits reverse thermal gelation properties. RTG systems are solutions, preferably substantially aqueous solutions, which are liquids at lower temperatures and transition to the gel state when at or above the gelation temperature. Preferred RTG systems comprise at least one gel-forming polymeric material; where the RTG systems comprise a blend of two or more gel-forming polymeric materials, the materials may be chosen to have different molecular weights, gelation temperatures and the like.

The terms “gel solution” and “blend” mean a substantially aqueous solution having a gel forming material, herein typically a cellulose derivative and/or gelatin, dissolved therein at a functional concentration, and maintained at a temperature above or below the gelation temperature such that gel formation does not occur. Gel solutions of the invention may be RTG systems.

By “substantially aqueous solution” herein is a solution that is water-based and optionally contains other water soluble liquids. Examples of other water soluble liquids include ethanol, propylene glycol, and low molecular weight polyethylene glycols (PEG), i.e., PEGs that are do not contribute to the reverse thermal gelation properties of the RTG system. Preferred substantially aqueous solutions are those that comprise, as a solvent, at least 50% water; more preferred substantially aqueous solutions are those that comprise, as a solvent, at least 75% water; even more preferred substantially aqueous solutions are those that comprise, as a solvent, at least 95% water; and particularly preferred substantially aqueous solutions are those that consist only of water as the solvent. The “continuous aqueous phase” of the invention is a substantially aqueous solution.

“Biodegradable polyesters” refers to any biodegradable polyester, which is preferably synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydrooxybutyric acids, malic acid, and copolymers thereof.

“Reverse thermal gelation” (“RTG”) is the phenomena whereby a solution, typically a substantially aqueous solution, of material, e.g., a cellulose derivative such as hydroxypropyl cellulose, ethyl cellulose, carboxycellulose, or methylcellulose, spontaneously increases in viscosity, and in many instances transforms into a semisolid gel, as the temperature of the solution is increased above the gelation temperature of the material. For the purposes of the invention, the term “gel” includes both the semisolid gel state and the high viscosity state that exists when gelation conditions are met. When cooled below the gelation temperature, the gel spontaneously reverses to reform the lower viscosity solution. This cycling between the solution and the gel may be repeated because the sol/gel transition does not involve any change in the chemical composition of the polymer system. All interactions to create the gel are physical in nature and do not involve the formation or breaking of covalent bonds.

“Microparticle-agent delivery liquid”, “microparticle-agent delivery liquid having reverse thermal gelation properties” or “microparticle-agent delivery liquid having stimuli responsive gelation environment” shall mean a polymer solution that contains a microparticle carrying an agent to be delivered, e.g. a drug (the agent per se can either be dissolved or colloidal) suitable for administration to a warm-blooded animal which forms a gelled microparticle/drug depot when the temperature is changed, depending upon the properties of the polymer, to above or below the gelation temperature of the block copolymer, or when other gelation conditions are met.

“Depot” means a gel formed from a microparticle-agent delivery liquid following its administration to a warm-blooded animal when the temperature has been changed, depending upon the properties of the polymer, to above or below the gelation temperature or when other gelation conditions are met.

### Dual Phase Polymeric Agent-Delivery Composition

The invention comprises a blend that forms a hydrogel and a microparticle system wherein the microparticle system is embedded in the hydrogel matrix or suspended in the blend. The hydrogel or blend comprises a cellulose derivative and a polyethylene glycol. One or more agents to be delivered may be located in the microparticle alone or both in the microparticle and the hydrogel matrix. The microparticle-hydrogel delivery system of the present invention provides for sustained release of the agent, and the release can be controlled so that it takes place at a relatively constant rate. The release profile of the system can be modified by altering the microparticle and/or the gel composition.

Prior to gelling, the blend, which may be referred to herein as the gel solution, is slightly more viscous than normal saline. Therefore, it is an excellent suspending agent for microparticles. The suspension of microparticles in the blend can be injected smoothly without clogging using a relatively small-gauge needle. After injection, the blend sets and forms a hydrogel and localizes the microparticle suspended in it. The agent encapsulated in the microparticle must be released from the microparticle before traveling through the hydrogel matrix and entering the biological system. Therefore, any immediate release, or burst, associated with microparticle delivery systems can be reduced and modulated. Since the release rates of the agent from these two systems can be quite different, embedding microparticles in the gel phase offers additional modulation and economical use of the agent. The benefits include longer duration of action than either system when used alone. Moreover, the combined system can improve the safety of microparticle dosage form. Since the microparticles are localized by the gel, they can be surgically retrieved, should one decide to terminate the medication delivery for any reason.

Artificial or natural microparticles including microcapsules, microspheres, and nanospheres contain the bio-active agent to be delivered to the body. Any bio-active agent having the ability to be contained and released by these microparticles may be used. The microparticles are then incorporated into the cellulose derivative/polyethylene glycol gel that is capable of releasing the microparticles and/or bioactive material within the biological environment, in a controlled manner.

The blends and hydrogels of the invention comprise saline, and preferably a phosphate buffered saline (PBS). The preferred saline for use in the invention is typically normal saline which has a sodium chloride concentration of about 0.9% w/v or 0.15 M. Suitable phosphate buffered saline for use herein may be any of a number of PBS variations which are well known in the art. A typical, suitable PBS for use herein has a sodium chloride concentration of 0.137 M, a potassium chloride concentration of 0.0027 M, a phosphate concentration of 0.010 M, and a pH of 7.4.

In the compositions of the invention, the cellulose derivative of the blend or gel solution is an alkylcellulose, a carboxyalkylcellulose, or a hydroxyalkylcellulose. Thus, cellulose derivatives of the invention include those where hydrogen atoms in the hydroxy (OH) groups of cellulose have been replaced by alkyl, such as methyl, ethyl, or propyl groups, hydroxyalkyl, such as hydroxyethyl or hydroxypropyl groups, benzyl or hydroxy-benzyl groups or alkalimetal salts of carboxymethyl, such as sodium acetate, or mixtures of these. Each glucose unit of cellulose may contain from 0-3 hydroxy groups substituted with above mentioned groups.

The amount of the cellulose derivative in the compositions and blends of the invention is from about 3% to about 12% by weight of the continuous (aqueous) phase. Because none of the components react or are otherwise reduced during the gelling of the blends, the amount of the cellulose derivative (as well as other components) remains the same in the gelled compositions. Preferred amounts of the cellulose derivative are from about 7% to about 9% by weight of the aqueous phase.

In addition to the cellulose derivative, the blends contain a polyethylene glycol. Suitable polyethylene glycols are those having a molecular weight of from about 3000 to 20,000. Preferred polyethylene glycols are those having a molecular weight of from about 3000 to 15,000. More preferred polyethylene glycols are those having a molecular weight of from about 3000 to 12,000. Particularly preferred PEGs are those having a molecular weight of from 3000 to 9000. One suitable example of a particularly preferred PEG has a molecular weight of from about 3000 to 8000.

The amount of the polyethylene glycol in the blends is from about 3% to about 12% by weight of the continuous (aqueous) phase. Preferably, the amount of the polyethylene glycol is from about 7% to about 12% by weight of the aqueous phase.

Particularly preferred blends are those where the amount of the cellulose derivative is from about 7% to about 9% by weight of the aqueous phase and the amount of the polyethylene glycol is from about 7% to 9%.

In certain aspects, the aqueous phase will also contain gelatin. The gelatin may be obtained from any source, and may be mammalian gelatin such as bovine-derived gelatin or fish gelatin. A preferred gelatin is obtained from bovine skin.

When used, the amount of gelatin in the aqueous phase is from about 0.25 to about 20% by weight. Preferably the amount of gelatin is from about 0.5 to 15% by weight of the aqueous phase. More preferably, the amount of gelatin is from about 1 to 15% by weight of the aqueous phase.

Preferred hydrogels of the invention retain their mechanical integrity for extended periods of time, typically for at least 8 weeks in phosphate buffered saline.

Upon administration to a mammal, the composition forms a gel and forms a depot, trapping the microparticles along with any agent or drug incorporated therein. Additional agents may optionally be contained in the microparticle and/or gel matrix.

The microparticles of the present invention may be microcapsules, microspheres, or nanospheres, currently known in the art, so long as they are capable of being entrained intact within a polymer that is or becomes a gel once inside a biological environment.

Preferred microparticles for use herein are microcapsules or microspheres. Preferred microparticles are also biodegradable.

Compositions of the invention contain from about 0.01 to about 30% by weight of microparticles, based on the total weight of the dual phase polymeric agent-delivery composition. Preferably, the amount of microparticles in the dual phase composition is from about 5-15% by weight, and more preferably from about 7-12% by weight.

The microparticles of the present invention comprise a solid polymer matrix with a biological active agent(s) dispersed or encapsulated within the matrix. These polymers can be non-biodegradable or biodegradable. Non-biodegradable but biocompatible polymers include silicone rubber, polyethylene, poly(methyl methacrylate) (PMMA), polystyrene (PST) ethylene-vinyl acetate copolymer (EVA), polyethylene-maleic anhydride copolymers, polyamides and others. Although these polymers may be effective, they remain in the body after exhaustion of the biologically active agent. When necessary, they must be surgically removed.

Conversely, when using biodegradable and/or absorbable polymers as the carrier, the carrier is gradually degraded or absorbed in the body simultaneously with or subsequent to the release of the biologically active agent. Therefore, it is preferred that a biodegradable copolymer is used in the present invention. Suitable biodegradable polymers for use herein include biodegradable polyesters such as polylactides, poly(D,L-lactide-co-glycolide)s, polyglycolides, poly(lactic acid)s, poly(glycolic acid)s, poly(D,L-lactic acid-co-glycolic acid)s, poly-ε-caprolactone), poly(hydroxybutyric acid), and poly(amino acid)s, polyorthoesters, polyetheresters, polyphosphazines, polyanhydrides, polyesteramides, poly(alkyl cyanoacrylate)s, and blends and copolymers thereof. Preferred polymers include biodegradable polyesters or polyester copolymers. More preferred polymers for use in the invention include poly(D,L-lactide-co-glycolide) with molecular weight between 5,000 to 70,000 Daltons with a lactide-to-glycolide ratio of about 1:1 to 1:0. The polymer end groups can be capped or uncapped with low molecular weight organic radicals. Preferred microparticles are microspheres comprising poly(D,L-lactide-co-glycolide) made using from about 45-80 mol % lactic acid and about 20-55 mol % glycolic acid. More preferred microparticles are microspheres comprising poly(D,L-lactide-co-glycolide) made using about 75 mol % lactic acid and about 25 mol % glycolic acid. Other more preferred microparticles are microspheres comprising poly(D,L-lactide-co-glycolide) made using about 50 mol % lactic acid and about 50 mol % glycolic acid.

Many microencapsulation techniques used to incorporate a bio-active agent into a microparticle carrier are taught in the art. See, for example, U.S. Pat. Nos. 4,652,441, 5,100,669, 4,438,253, and 5,665,428, each of which is incorporated herein by reference in its entirety. Commonly employed methods include: (a) phase separation and subsequent organic solvent evaporation (include O/W emulsion, W/O emulsions, O/O′ emulsions and W/O/W emulsions), (b) coacervation, (c) melt dispersion; (d) spray drying, (e) spray congealing, (f) air suspension coating; and (h) pan coating.

Preferred microspheres for use in the invention include those made using a double emulsion technique at water:oil ratios of from about 25:1 to 50:1. More preferred microspheres for use in the invention include those made using a double emulsion technique at water:oil ratios of from about 30:1 to 40:1. Particularly preferred microspheres for use in the invention include those made using a double emulsion technique at water:oil ratios of from about 30:1 to 35:1. Microspheres of the invention are preferably those made to have loading efficiencies of from about 35-75%, and more preferably from about 45-60%, and even more preferably from about 50-55%.

Preferred microspheres for use in the invention include those having average diameters of from about 75-125 μm; more preferred microspheres of the invention are those having average diameters of from about 100-115 μm.

In certain aspects of the invention, other temperature sensitive biocompatible polymers (secondary gelling polymers) can be combined with the cellulose derivative/polyethylene glycol blend of the invention to form the gel matrix. Examples of such materials are described in U.S. Pat. No. 6,287,588, the disclosure of which is incorporated herein in its entirety. For example, a block copolymer having thermal gelation properties wherein the polymer is a gel at physiological temperatures (about 37° C.) and is a liquid above or below physiological temperatures would be suitable. In the case of a gel having reverse thermal-gelation properties, the block copolymer would be a liquid at temperatures below the gelation temperature and would form a gel at above the gelation temperature. Conversely, a block copolymer having conventional thermal-gelation properties would be a liquid above the gelation temperature and a gel at or below the gelation temperature.

Biocompatible polymers having reverse gelation properties are most preferred for use with the cellulose derivative/polyethylene glycol blends of the invention.

Biocompatible polymers exhibiting other properties may also be used with the cellulose derivative/polyethylene glycol blends of the invention. Other environmentally sensitive polymers may be used such as those that respond to changes in pH, ionic strength, solvent, pressure, stress, light intensity, electric field, magnetic field and/or specific chemical triggers such as glucose. The critical element is that the polymer be in a gel state for the period of time while within the body. Additionally, when considering what polymeric gels and/or microparticles to use, resistance to bioactive agent release is an important consideration. With some bioactive agent, where very prolonged and uniform release is desirable, gels and microparticles having a stronger and more uniform resistance (hence a more prolonged and uniform release of bioactive agent) should be selected. As such, polymeric gels and microparticles should be selected carefully based on how the bioactive agent is desired to be delivered.

One suitable secondary gelling polymer for use in the present invention comprises ABA- or BAB-type block copolymers, where the A-blocks are relatively hydrophobic A polymer blocks comprising a biodegradable polyester, and the B-blocks are relatively hydrophilic B polymer blocks comprising polyethylene glycol (PEG). The A block is preferably a biodegradable polyester synthesized from monomers selected from the group consisting of D,L-lactide, D-lactide, L-lactide, D,L-lactic acid, D-lactic acid, L-lactic acid, glycolide, glycolic acid, .epsilon.-caprolactone, ε-hydroxyhexanoic acid, γ-hydroxybutyric acid, δ-valerolactone, δ-hydroxyvaleric acid, hydroxybutyric acids, malic acid, and copolymers thereof, and the B block is PEG. In the most preferred embodiment, the A block is comprised of poly(D,L-lactide-co-glycolide) and the B block is PEG. Preferably, the triblock copolymer has an average molecular weight between 300 and 20000 Daltons and contains about 10 to 83% by weight of A block polymer. More preferably, the triblock copolymer has an average molecular weight between 500 and 5000 Daltons and contains about 51 to 83% by weight of A block polymer.

The secondary gelling polymer is preferably biodegradable and exhibits water solubility at low temperatures and undergoes reversible thermal gelation at physiological mammalian body temperatures. Furthermore, these polymeric gels are biocompatible and capable of releasing the substance entrained within its matrix over time and in a controlled manner. As such, this polymeric gel, or others having desired properties, may be used to control release of various microparticles as described above. These biodegradable polymers are gradually degraded by enzymatic or non-enzymatic hydrolysis in aqueous or physiological environments. The degradation products are polyethylene glycol, lactic acid and glycolic acid. These compounds are relatively innocuous and can easily be excreted or absorbed by the biological system.

An advantage of the delivery system of the invention lies in the ability of the drug/microparticle embedded polymeric gel to increase the chemical stability of many drug substances. The agent or drug can be released into the biological environment via either of two pathways. The drug contained in the microparticle can be released into the polymeric gel matrix first, and then released from the gel matrix to the target. Alternatively, the microparticles containing the drug can be released from the gel first, and then the drug encapsulated in the microparticles may be released to the target.

In addition, the various combinations of microparticle and gelling solution contributes to flexibility in designing drug delivery systems to meet particular situations. Thus, drug delivery systems can be made according to the invention to have release profiles modified by modulating the drug dissolution rate and gel matrix erosion rate.

The gelling solutions of the invention are typically prepared by first dissolving the cellulose derivative in water, preferably saline, and more preferably phosphate buffered saline, and subsequently adding the polyethylene glycol. Alternatively, the cellulose derivative may be added to a solution of polyethylene glycol in water. In another alternative, an aqueous solution of the cellulose derivative, preferably cooled to a temperature below its gel transition temperature, may be added to the polyethylene glycol. Regardless of which method is employed to make the blend, cooling and agitation will be necessary and should be used as appropriate. In general, cooling will be necessary to maintain the blend as a solution. In certain situations, the cellulose derivative may be added to warm water to avoid agglomeration and clumping of the cellulose derivative.

After preparation, the blend may be stored or combined immediately with the microparticles. Agitation or mixing may be necessary to uniformly distribute the microparticles throughout the blend. Dual phase systems of microparticles in the gelling solution can then be stored as necessary prior to use.

In another alternative, after preparation of the gel solution (but prior to combination with the microparticles), the solution may be dried or lyophilized to provide a powder that would require reconstitution with an aqueous vehicle. If all the solid components were used to make the gel solution, only water would be required for reconstitution. The gel solution formed by reconstitution would then be mixed with microparticles before administration.

An aqueous solution of drug/microparticle at a temperature below the gelation temperature forms a drug/microparticle delivery liquid where the drug may be either partially or completely dissolved. When the drug/microparticle is partially dissolved, or when the drug/microparticle is essentially insoluble, the drug-carrying microparticle exists in a colloidal state, such as a suspension or emulsion. This drug/microparticle delivery liquid is then administered parenterally, topically, transdermally, transmucosally, inhaled, or inserted into a cavity such as by ocular, vaginal, transurethral, rectal, nasal, oral, buccal, pulmonary or aural administration to a patient, whereupon it will undergo reversible thermal gelation, or other stimuli responsive gelation.

The main mechanism of in vivo degradation of the polymers is by hydrolysis and/or enzymatic degradation. The duration of sustained delivery can be adjusted from few days up to one year through proper selection of the durability of the gel and microparticle durability.

Release of the biologically active agent is usually tri-phasic. It comprises an initial burst or, immediate release of the agent present at or near the surface of the microparticle, a second phase during which the release rate is slow or sometime no bio-active agent is released, and a third phase during which most of the remainder of the biologically active agent is released as erosion proceeds. Any agent, as long as it is suitable for microencapsulation in a microparticle, as is known in the art, can utilize the delivery system described by the current invention.

Since the polymeric gel and/or microparticle of the delivery system of this invention are preferably biocompatible and biodegradable, there is minimal toxic effect and irritation to the host. The drug release profile can be controlled and improved by proper design and preparation of various gel forming polymers or copolymer blocks. The release profile of the polymeric gel may also be modified through preparation of a gel blend by selection of individual gel systems and ratios of individual gel systems in the blend. Drug release is also controllable through adjustment of the concentration of the gel blends in the drug delivery liquid. Although it is preferred in the present invention that a RTG system is used, a gel blend of two or more non-RTG with desired gelation properties is also within the scope of the present invention.

An additional or a second agent can also be loaded into the microparticles and/or the polymeric gel matrix. The second agent can be a regulatory agent for the microparticle and/or the gel, or a second bio-active agent to be released into the biological environment in a same or different release rate.

The only limitation as to how much drug can be loaded into the microparticle and how much of such drug carrying microparticle can be loaded into the polymeric gel is one of functionality, namely, the drug/microparticle load may be increased until the microparticle structure, and/or the gelation properties of the polymer or copolymer are adversely affected to an unacceptable degree, or until the properties of the system are adversely affected to such a degree as to make administration of the system unacceptably difficult. Generally speaking, about 0.0001 to 30% by weight of a drug can be loaded into a microparticle with 0.001 to 20% being most common. The drug carrying microparticle will generally make up between 0.0001 to 30% by weight of the formulation with ranges of between about 0.001 to 20% being most common. These ranges of drug/microparticle loading are not limiting to the invention. Provided functionality is maintained, drug loadings outside of these ranges fall within the scope of the invention.

This invention is applicable to delivery of bio-active agents and drugs of all types including oligonucleotides, hormones, anticancer-agents, and it offers an unusually effective way to deliver polypeptides and proteins. Many labile peptide and protein drugs are amenable to formulation into the microparticle and/or the gel polymer or block copolymers and can benefit from the reverse thermal gelation process described herein. While not specifically limited to the following, examples of pharmaceutically useful polypeptides and proteins may be selected from the group consisting of erythropoietin, oxytocin, vasopressin, adrenocorticotropic hormone, epidermal growth factor, platelet-derived growth factor (PDGF), prolactin, luliberin, luteinizing hormone releasing hormone (LHRH), LHRH agonists, LHRH antagonists, growth hormone (human, porcine, bovine, etc.), growth hormone releasing factor, insulin, somatostatin, glucagon, interleukin-2 (IL-2), interferon-α,β, or γ, gastrin, tetragastrin, pentagastrin, urogastrone, secretin, calcitonin, enkephalins, endorphins, angiotensins, thyrotropin releasing hormone (TRH), tumor necrosis factor (TNF), nerve growth factor (NGF), granulocyte-colony stimulating factor (G-CSF), granulocyte macrophage-colony stimulating factor (GM-CSF), macrophage-colony stimulating factor (M-CSF), heparinase, bone morphogenic protein (BMP), hANP, glucagon-like peptide (GLP-1), interleukin-11 (IL-11), interleukin-12(IL-12),VEG-F, recombinant hepatitis B surface antigen(rHBsAg), renin, bradykinin, bacitracins, polymyxins, colistins, tyrocidine, gramicidins, cyclosporins and synthetic analogues, modifications and pharmacologically active fragments thereof, enzymes, cytokines, antibodies and vaccines.

The only limitation to the polypeptide or protein drug which may be utilized is one of functionality. In some instances, the functionality or physical stability of polypeptides and proteins can also be increased by the addition of various additives to aqueous solutions or suspensions of the polypeptide or protein drug. Additives, such as polyols (including sugars), amino acids, surfactants, polymers, other proteins and certain salts may be used. These additives can readily be incorporated into the microparticle/polymer gel system of the present invention, which will then undergo a gelation process.

Developments in protein engineering may provide for the possibility of increasing the inherent stability of peptides or proteins. While such resultant engineered or modified proteins may be regarded as new entities in regards to regulatory implications, that does not alter their suitability for use in the present invention. One of the typical examples of modification is PEGylation, where the stability of the polypeptide drugs can be significantly improved by covalently conjugating water-soluble polymers such as polyethylene glycol with the polypeptide. Another example is the modification of the amino acid sequence in terms of the identity or location of one or more amino acid residues by terminal and/or internal addition, deletion or substitution. Any improvement in stability enables a therapeutically effective polypeptide or protein to be continuously released over a prolonged period of time following a single administration of the drug delivery liquid to a patient.

In addition to peptide or protein based drugs and bioactive agents, any other agents needed to be delivered into an desired environment in controlled manner for a extended period, may be utilized in the present system; e.g, a food releasing system in a fish tank, or fertilizer/nutritional releasing system. Again, the only limitation is the compatibility between the agent and the microparticle and the polymeric gel.

As noted above, the invention provides methods for treating or repairing a joint in a mammal comprising injecting into joint space in need of such treatment a composition of claim 1. Specific examples of the disorders that may be treated with the invention include arthritis, and in particular osteoarthritis and rheumatoid arthritis. In this aspect, the microparticles, preferably microspheres, are made to contain a drug or material useful in treating joint diseases or disorders. Among these are anti-interleukin and anti-TNF antibodies, glucosamine, chondroitin sulfate, and hyaluronic acid.

In addition, the compositions of the invention can be used to deliver pharmaceutical agents for use in treating traumatic cartilage injuries, tendon/ligament rupture, and bone fractures. In these methods the compositions could deliver agents for tissue regeneration.

Further, the compositions can be used to treat infections in patients. It will be appreciated that the invention is particularly well adapted for localized delivery of anti-infective agents to patients having a local infection or a localized infection. Thus, the invention can be used to prevent or treat infections in, for example, burn patients.

Further, the compositions of the invention can be used in the treatment of cancer. In this aspect, the invention provides methods of localized delivery of anti-tumor agents. The anti-tumor agent can readily be delivered in an effective amount to an area or organ in a patient that requires treatment. The invention efficiently delivers the agent without requiring systemic delivery.

The present invention may be further illustrated by reference to the following examples.

### EXAMPLE 1

### Methylcellulose/Polyethylene Glycol Hydrogel

Phosphate buffered saline (PBS) is added to polyethylene glycol (PEG) powder in a suitable vessel and the resulting mixture is agitated to dissolve the PEG. The mixture may be heated as necessary, e.g., to 37° C. or 60° C., to accelerate dissolution.

Methylcellulose (MC) is then added to the solution of PEG in PBS and the mixture is agitated, vigorously if necessary, to dissolve the methylcellulose. The mixture is alternately agitated and cooled (ice bath), e.g., for periods of about 5 minutes each, until the methylcellulose has completely dissolved and the mixture is a uniform blend of the PEG and methylcellulose. The blend may then sonicated to remove any gas bubbles and then may be stored at about 4° C. prior to use.

The following Methylcellulose/Polyethylene glycol blends can be prepared essentially as described above in this example.

PBS/MC/PEG Blends

| | Ingredient | | |
|---|---|---|---|
| Blend | (% by weight of blend) | | |
| No. | MC | PEG 3400 | PEG 7500 |
| 1 | 5 | 4 | — |
| 2 | 5 | 6 | — |
| 3 | 5 | 8 | — |
| 4 | 6 | 4 | — |
| 5 | 6 | 6 | — |
| 6 | 6 | 8 | — |
| 7 | 7 | 4 | — |
| 8 | 7 | 6 | — |
| 9 | 7 | 8 | — |
| 10 | 5 | — | 4 |
| 11 | 5 | — | 6 |
| 12 | 5 | — | 8 |
| 13 | 6 | — | 4 |
| 14 | 6 | — | 6 |
| 15 | 6 | — | 8 |
| 16 | 7 | — | 4 |
| 17 | 7 | — | 6 |
| 18 | 7 | — | 8 |

### EXAMPLE 2

### Methylcellulose/Polyethylene Glycol/Gelatin Hydrogel

A blend is prepared to contain MC, PEG, and gelatin. The procedure is essentially as described above in Example 1 but gelatin is dissolved in the PBS with the polyethylene glycol at 60° C. After addition of the MC, the mixture is alternately agitated and cooled (ice bath) for 10 minute periods.

The following Methylcellulose/Polyethylene glycol/gelatin blends can be prepared essentially as described above in this example.

PBS/MC/PEG/Gelatin Blends

| | Ingredient | | |
|---|---|---|---|
| Blend | (% by weight of blend) | | |
| No. | MC | PEG 7500 | Gelatin |
| 19 | 7 | 6 | 1 |
| 20 | 8 | 6 | 1 |
| 21 | 9 | 6 | 1 |
| 22 | 7 | 4 | 1 |
| 23 | 8 | 4 | 1 |
| 24 | 9 | 4 | 1 |
| 25 | 7 | 8 | 1 |
| 26 | 8 | 8 | 1 |
| 27 | 9 | 8 | 1 |

### EXAMPLE 3

Poly(D,L-lactide-co-glycolide) microspheres (75% lactic acid, 25% glycolic acid, molar basis) are prepared essentially according to the procedures set forth in U.S. Pat. No. 5,674,534 to contain 1.66 mg chondroitin sulfate per 100 mg of microspheres. 500 mg of these microspheres are added to 5 ml of the methylcellulose/polyethylene glycol/gelatin blend No. 20 of Example 2 and agitated to distribute the microspheres uniformly throughout the blend. The microsphere/blend mixture is stored at 4° C.

Aliquots (1 ml) of the microsphere/blend mixture are added to wells of a 12-well plate and the plate is incubated at 37° C. for 30 minutes. The blend forms a hydrogel with entrapped microspheres containing chondroitin sulfate.

### EXAMPLE 4

Poly(D,L-lactide-co-glycolide) microspheres (50% lactic acid, 50% glycolic acid, molar basis) are prepared essentially according to the procedures set forth in U.S. Pat. No. 5,674,534 to contain 1.66 mg chondroitin sulfate per 100 mg of microspheres.

To 5 ml of a heated solution of polyethylene glycol in phosphate buffered saline (8% by weight PEG) is added methylcellulose in an amount sufficient to make a solution containing 8% by weight methylcellulose. The resulting mixture is agitated. After all solids have dissolved, 500 mg of the 50:50 chondroitin sulfate microspheres prepared above are added to the polyethylene glycol/methylcellulose blend, agitated to uniformly distribute the microspheres, and subsequently stored at 4° C.

Aliquots (1 ml) of the microsphere/blend mixture are added to wells of a 12-well plate and the plate is incubated at 37° C. for 30 minutes. The blend forms a hydrogel with entrapped microspheres containing chondroitin sulfate.

### EXAMPLE 5

### Microsphere Preparation

A double emulsion technique is employed to encapsulate chondroitin sulfate (CS) in 50:50 PLGA microspheres. 50 mg of CS is dissolved in phosphate buffered saline (PBS), and then emulsified in a solution of 1.25 g of PLGA in 10 ml methylene chloride. This mixture is added to 0.1% polyvinyl alcohol to form the second emulsion. The methylene chloride is extracted through an evaporation step using 2% isopropanol and the microspheres are collected and washed. The average diameter of the microspheres is calculated to be 107±36.5 μm. Microspheres prepared in this example have water:oil ratios of about 33:1.

### Microsphere Loading Efficiency and CS Release

To determine CS loading efficiency, 100 mg of microspheres are degraded overnight at 37° C. in 1 M NaOH, and the CS content encapsulated in the microspheres is evaluated using a Blyscan glycosaminoglycan assay. Microspheres prepared in this example have loading efficiencies of about 52% (52.4±8.52%).

Half of the remaining microspheres are embedded in thermal-sensitive MC-PEG hydrogels, which are prepared by dissolving MC in a heated aqueous solution of PEG and then chilled at 4° C. overnight. The hydrogel is allowed to congeal at 37° C. in a tissue culture insert of a 12-well plate.

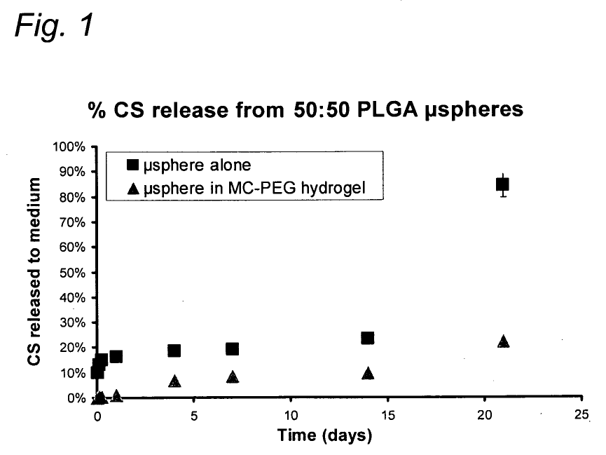

In triplicate, 100 mg of microspheres and 1 ml of MC-PEG hydrogels containing 100 mg of microspheres are separately placed in solutions of PBS and 0.2% NaN₃ and release of CS from the microspheres is measured over a period of 3 weeks. The results of the CS Release assay are shown in FIG. 1, which is a graph comparing chondroitin sulfate (CS) release from 50:50 PLGA microspheres alone with CS release from 50:50 PLGA microspheres embedded within the MC-PEG hydrogel prepared above in this example.

An initial burst release of CS is observed from microspheres alone, with 17% of the bioactive agent lost to the medium after the first day. CS release rate then drops sharply, with 19.3% and 23.4% released after 1 week and 2 weeks, respectively. A second large release of CS is then observed at 3 weeks as 84.2% of the drug is measured in the medium. Also at this stage, the microspheres appear shrunken and partially degraded. By contrast, only 0.8% of the CS is released from the microspheres embedded in hydrogels within 24 hours, followed by a quasi-linear release thereafter. After 3 weeks, 78.0% of the total theoretical amount of CS inside the microspheres embedded in the hydrogel remained.

### Biocompatibility

Finally, to examine the biocompatibility of the microspheres and hydrogels, bovine articular chondrocytes at the second passage are seeded at the bottom of a 12-well plate in three groups. Cells are either exposed to ethylene-oxide sterilized microspheres, microspheres incorporated into sterilely prepared MC-PEG hydrogels, or left unexposed for 3 days. Cytotoxicity is evaluated according to the criteria described in the ISO 10993 protocol for Biocompatibility Evaluation. The biocompatibility tests show that chondrocytes proliferate well during exposure to microspheres and microspheres embedded in hydrogels. No substantial differences are observed in the morphology or confluence of the cells in any of the groups. All cells appeared elongated, exhibiting a fibroblastic morphology at 80% confluence. Neither cell detachment nor round-up is observed in any culture conditions.

While the invention has been described with reference to certain preferred embodiments, those skilled in the art will appreciate that various modifications, changes, omissions, and substitutions can be made without departing from the spirit of the invention. It is intended, therefore, that the invention be limited only by the scope of the following claims.

What is claimed is:

1. A dual phase polymeric agent-delivery composition comprising:
(a) a continuous aqueous phase comprising a reverse thermal gelation system comprising a blend of a cellulose derivative and polyethylene glycol;
(b) a discontinuous particulate phase comprising microparticles; and
(c) an agent to be delivered contained in at least said discontinuous particulate phase.

2. A composition according to claim 1, wherein the cellulose derivative is an alkylcellulose, a carboxyalkylcellulose, or a hydroxyalkylcellulose.

3. A composition according to claim 1, wherein the cellulose derivative is hydroxypropylcellulose, hydroxyethylcellulose, hydroxymethylcellulose, methylcellulose, ethylcellulose, or propylcellulose.

4. A composition according to claim 1, wherein the cellulose derivative is methylcellulose or ethylcellulose.

5. A composition according to claim 3, wherein the polyethylene glycol has a molecular weight of from about 3000 to 15,000.

6. A composition according to claim 5, wherein the polyethylene glycol has a molecular weight of from about 3500 to 10,000.

7. A composition according to claim 6, wherein the polyethylene glycol has a molecular weight of from about 3500 to 8000.

8. A composition according to claim 3, wherein the amount of the cellulose derivative is from about 3% to about 12% by weight of the aqueous phase.

9. A composition according to claim 8, wherein the amount of the cellulose derivative is from about 7% to about 9% by weight of the aqueous phase.

10. A composition according to claim 1, wherein the aqueous phase comprises saline.

11. A composition according to claim 3, wherein the amount of the polyethylene glycol is from about 3% to about 12% by weight of the aqueous phase.

12. A composition according to claim 11, wherein the amount of the polyethylene glycol is from about 7% to about 9% by weight of the aqueous phase.

13. A composition according to claim 3, wherein the amount of the cellulose derivative is from about 7% to about 9% by weight of the aqueous phase and the amount of the polyethylene glycol is from about 7% to about 9%.

14. A composition according to claim 13, where the aqueous phase comprises gelatin.

15. A composition according to claim 14, wherein the amount of gelatin in the aqueous phase is from about 0.25% to about 2% by weight.

16. The composition according to claim 1 wherein the microparticles are suspended in the aqueous phase and are in the form of microcapsules, microspheres, or nanospheres.

17. The composition according to claim 16 wherein the microparticle is in the form of microcapsules or microspheres.

18. The composition according to claim 1 wherein the microparticle is biodegradable.

19. A composition according to claim 1, wherein the agent is an anti-interleukin or anti-TNF antibody, glucosamine, chondroitin sulfate, or hyaluronic acid.

20. A composition according to claim 1 wherein the microparticle content of the composition is between about 0.01% and about 30% by weight.

21. A method of treating or repairing an articulating joint defect comprising injecting into the joint space a composition of claim 1.

22. A method of treating osteoarthritis comprising administering to a joint displaying symptoms of osteoarthritis an effective amount of a composition of claim 1.

23. A method for delivering an agent to a mammalian subject in a controlled manner for a sustained period of time, comprising:
(a) providing a dual phase polymeric delivery composition according to claim 1,
(b) maintaining said composition as a liquid;
(c) administering said composition as a liquid to a confined location in the patient; and
(d) permitting the composition to form a gel within the confined location in the patient.

24. A method according to claim 23, wherein the composition is administered to the confined space by injection.

25. A method according to claim 24, wherein the confined space is an articulating joint in the patient.

26. A dual-phase sustained release gelled dosage form comprising
(a) a continuous hydrogel phase comprising a blend of a cellulose derivative and polyethylene glycol;
(b) a discontinuous particulate phase comprising microparticles; and
(c) an agent to be delivered contained in at least said discontinuous particulate phase.

27. A gelled dosage form according to claim 26, wherein the hydrogel phase is a reverse thermal gelation system.
